(19) United States
(12) Patent Application Publication
(10) Pub. No.: US 2024/0073668 A1
(43) Pub. Date: Feb. 29, 2024

(54) Big Data 911TM: Tracking Phones and/or Vehicles to Ensure Safety

(71) Applicant: Mark Haley, McKinney, TX (US)

(72) Inventor: Mark Haley, McKinney, TX (US)

(21) Appl. No.: 18/193,021

(22) Filed: Mar. 30, 2023

Related U.S. Application Data
(63) Continuation-in-part of application No. 17/302,264, filed on Apr. 29, 2021.

(30) Foreign Application Priority Data
Aug. 30, 2022 (JP) 2022-136667

(51) Int. Cl. H04W 4/90; A61B 5/00; A61B 5/11; A61B 5/117; G16H 10/65

(52) U.S. Cl. CPC H04W 4/90; A61B 5/1112; A61B 5/117; A61B 5/4842; A61B 5/747; G16H 10/65

(57) ABSTRACT

Device, system and method, for simplicity call Big Data 911™, which expedites response times after vehicle accidents or other emergencies. It automatically initiates a 911 computer call from a vehicle and/or a phone with data comprising GPS data location, speed, etc., Caller ID and the person's preapproved medical history all of which is continuously linked to the cloud before and/or after the crash. This data indicates when a crash and/or other emergency-occurs using speed, acceleration and deceleration and other data. Multiple sources of data when available offers redundancy to verify data and avoid unnecessary 911 calls. It expedites transportation to the hospital and ensures the optimum medical treatment customized for the victim during the crucial Golden Hour after the accident. This solution can be used nationally or internationally using Big Data in worldwide networks which track billions of phones and/or vehicles.

This application is a continuation-in-part application of and claims priority to U.S. patent application Ser. No. 17/302,264 entitled “Big Data 911™—EXPEDITED EMT RESPONSE AFTER VEHICLE ACCIDENTS OR OTHER EMERGENCIES”, filed on Apr. 29, 2021, the disclosure of which is incorporated herein by reference in its entirety.

### TECHNICAL FIELD

Device, system, and method to expedite response times after vehicle accidents or other emergencies by automatically initiating a 911 computer call from a vehicle and/or a phone with data comprising GPS location, speed, etc. and the person's preapproved medical history which is continuously linked to the cloud. A vehicle's onboard cameras, sensors, and computers, and/or a phone's sensors, computer, etc. indicate when a crash occurs using speed, deceleration, and other data. Using multiple sources of data when available offers redundancy to verify data and avoid unnecessary 911 calls. It expedites transportation to the hospital and ensures the optimum medical treatment customized for the victim during the crucial Golden Hour after the accident. This solution can be used nationally or internationally using Big Data worldwide networks which track billions of phones and/or vehicles.

### BACKGROUND

Device, system, and method which expedite Emergency Medical Technicians (EMTs), police, etc. response after Vehicle Accidents or Other Emergencies anywhere, such at home or in vehicle crashes, thereby ensuring the ideal medical care after accidents. There were 36,560 U.S. traffic deaths in 2018 and faster emergency team response times improves the chances for survival of hundreds or even thousands of accident victims each year. In 2018, 10% of fatal crashes were reported in over ten minutes. This-identifies technologies and economic incentives so that drivers permit sharing crash data automatically during the seconds leading to an accident. The GPS data, medical histories, and other data is continuously connected and to and stored in the cloud and in a millisecond once a crash and/or other emergency is detected an automatic call is made to the 911 network either with a text to voice link or with all the relevant info displayed on the 911 call center screens. The vehicle and/or phone send GPS data in a continuous link to the enhanced 911 cloud network. Using standard GPS data (sample below), with as little as 100 characters comprising the latitude, longitude, elevation and with as little as once per second, this pinpoints the location and speed of the tracker and also confirms the type of vehicle and when a crash occurs.

This amazing patent only requires a communication device which collects and sends GPS data, such as a smartphone, or ideally a satellite phone. This device would send a distress text or 911 call whenever that person travels worldwide on any vehicle, i.e. a car, aircraft, train etc. using GPS to detect a crash. Other systems are expensive and require sensors on the vehicle, such as GM's OnStar or others which use sensors such accelerometers to precisely determine when crashes occur and deploy airbags, call 911, etc. Our system only requires GPS data which determines the type of vehicle and when an emergency occurs. While Mark Haley was a Professor in Japan in 2009 and its part of his patent and for over 13 years, he has enhanced this GPS tracking technology with communications to train and track Skydivers worldwide, including elite Smokejumpers who skydive into remote fires. This technology is extremely cost effective because any device with GPS and communication, i.e. a smartphone, can track and protect people worldwide and send out distress text or calls in an emergency. As a result, the world's billions of phones (i.e. smart phones, satellite phones etc.) provide Big Data 911 safety for anyone traveling worldwide in any vehicle such as a car, motorcycle, train, aircraft, boat, etc.
Sample Standard GPS—$GPRMC,235316.000,A,4003.9040,N,10512.5792,W,0.09,144.75,141112,*19

One GPS data point provides the location, such as Dallas, Texas. Two GPS data points give speed, such as 88 feet/sec, or 60 mph, as the distance/time traveled. Three points detail acceleration/deceleration. In a head-on collision with a cement wall there'd be very little movement from the 2^{nd} to the 3^{rd} data point. The crash would be verified by cross-checking historical data for that type of vehicle. Redundancy of data, such as cell phone tracker, with a vehicle tracker, would reduce false 911 calls. The type of vehicle is identified by the speed and elevation data as shown in FIG. 7-11. After the crash, a text message with all relevant location, crash data and medical records is sent to 911. In addition, an optional text-to-speech (such as IBM's Watson) could be made in any language. However, the text message alone provides the ideal summary of the crash.

A vehicle automatically identifies its type of vehicle. However, a smart phone can only determine the type of vehicle using GPS data cross checked with historical data to confirm when a person is in a car, train, plane, even when the person jumps out of the plane and skydives. The author used this technique to track hundreds of trains, planes, cars and skydives and in accident investigations. While larger GPS databases provide more precision, even a small database of GPS data identifies the type of vehicle. The method and system use one or more sensors, including GPS data, on vehicles and/or phones to automatically make 911 calls when needed (FIG. 1-6). Redundancy of sensors reduces the chance of an incorrectly calling 911. However, this method and system has enough information using only the GPS data from a smart cell-phone to make a 911 call (FIG. 7).

In 2018, only 10% of U.S. drivers permitted sharing telematic vehicle data, the key info on the vehicle's speed, location, acceleration, etc. However, with larger automobile insurance discounts, more privacy protections and better technology, more drivers could be enticed to share this data thereby expediting response times and improving the chances for survival of accident victims. The European Union is far ahead of the US since after April 2018 all European Car Makers have been required to include eCall, an automated emergency call technology.

Big Data is the rapid analysis of massive amounts of data to solve preciously unsolvable problems. Big Data 911™ harnesses data from the vehicle and/or phone, the cloud plus the key info which is voluntarily disclosed by passengers linked to the cloud and data from a vehicle's cameras and sensors which monitor its speed, location, and identifies the driver using onboard cameras and facial recognition software (FIGS. 1-3). A vehicle generates terabytes of data per day and millions of vehicles must be tracked. Key crash data is automatically sent to 911 even from remote locations, or at night, where there aren't manual 911 calls. These robotic calls identify the location and severity of accidents, the driver's medical history, etc. helping save victims in the Golden Hour after crashes when the chances of survival are the highest. Also, by constantly monitoring vehicles, even when there is no data, such as failures of onboard sensors or loss of communications, this indicates potential problems or accidents (FIG. 4). This system works nationally and internationally (FIG. 5).

Big Data 911™ expedites the response times of first responders after traffic accidents thereby potentially saving lives or reducing injuries. On crowded urban U.S. roads many people immediately call 911 after an accident. Some cars even automatically send out distress calls. However late at night and in remote areas, the accident may not be reported for hours. Enhanced Big Data and the cloud automatically report those accidents during the seconds leading up to a crash, rather than 10 minutes after the accident.

Today's newest car sensors collect terabytes of data each day some of which are used to autonomously drive the car or even brake in potential accidents. This device, system and method invention focuses on ensuring that key parts of this data is sent to the cloud to alert first responders immediately after an accident.

“Golden Hour”—It's Crucial to Expedite Treatment within an Hour to Save Lives

Per the American College of Surgeons, the idea of the “Golden Hour” highlights the crucial need to successfully treat a patient in the first 60 minutes after a major injury such as an automobile crash or a gunshot wound. The method of treating trauma is called the “Advanced Trauma Life Support (ATLS)”. It was developed in 1976 based on experiences treating those seriously injured during the Vietnam War and in dangerous U.S. cities.

### BRIEF DESCRIPTION OF THE DRAWINGS

FIG. 1 Big Data and 911 Expedites Emergency Medical Care (Overview)

FIG. 2 Sample Sensors/Cameras in 2021 Autonomous Vehicles

FIG. 3 eCall is a good 1^{st} Step the 2^{nd} step is Big Data and Cloud Tracking

FIG. 4 Big Data and Cloud Monitors Drivers even with no Cell Network

FIG. 5 GPS/other Data from Millions of Vehicles/Phones sent to the Cloud Optimizing Emergency Response

FIG. 6 Emergency Network Tracking using only GPS and/or GPS and other Data

FIG. 7 Flow Chart showing how flight data is cross-checked to automatically identify the type of activity (plane, skydive) for 3D interactive plotting and best angles for debriefings.

FIG. 8 Flow Chart reveals the logic to dynamically plot customized 3D Plotting/Debriefings for any type of movement in 3D interactive Graphics.

FIG. 9 Flow chart logic for creating customized 3D plotting/debriefings.

FIG. 10 Flow chart logic for creating customized 3D plotting/debriefings.

FIG. 11 Flow chart logic for creating customized 3D plotting/debriefings.

FIG. 12 Planes, Skydives, Automobiles—Tracking all the time, anywhere

### DETAILED DESCRIPTION

This device, system and method using Big Data and the Cloud offers an integrated method of responding to vehicle accidents or general 911 calls so that EMTs, police, etc. know the medical background of the victims(s) and provide appropriate treatment immediately and during the transport to the hospital. The goal is to expedite moving the victim to the hospital and to provide the ideal medical treatment in the Golden Hour after an accident to ensure the best medical outcome. For vehicle accidents, a device, ideally mandated by the government similar to the mandate for smoke alarms, would automatically send out a distress call to 911 as the vehicle data indicated an impeding crash. This call would be just prior to impact in case it is a massive wreck which disables all communications and/or on-board computers. The information includes the identity of the driver using cameras plus facial or voice recognition software which monitors the driver as part of the autonomous driving system for the latest vehicles. Alternatively, in case the system fails to identify the passenger(s), and due to privacy concerns, an option to voluntarily provide medical records into the vehicle's computers or phone provides the medical history the passenger wants to disclose for accidents.

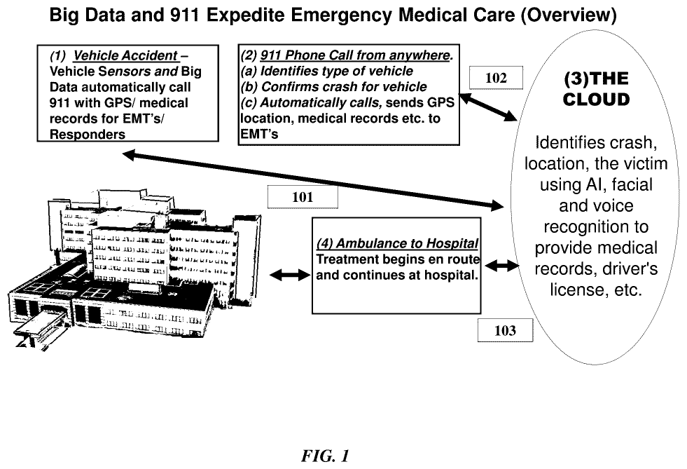

In FIG. 1, Block 1, a vehicle's sensors, computers, and software automatically call 911 with the GPS data and medical records for EMT's/Responders. If it's a 911 call from a phone it also automatically sends GPS data and/or Voice recognition identifies medical records and sends the GPS location and medical records to EMT's.

Using voice or facial recognition software calls or voluntarily uploaded data plus cross checking with cloud data in either a vehicle accident or a 911 call from home, the location, the identity, and medical background of the victim is known. Therefore, the EMTs or other responders can customize the treatment of the patient in route to the hospital. Block 3—using facial or voice recognition software or caller ID info, or voluntary disclosed information from a phone app linked to the vehicle's computers, the victim's medical records are available from Big Data in the cloud with Artificial Intelligence (AI) constantly reviewing the appropriate medical issues and treatments. While being transported to the hospital, Block 4, this system has identified the patient with related medical histories so the EMTs can provide initial customized treatment which would continue at the hospital.

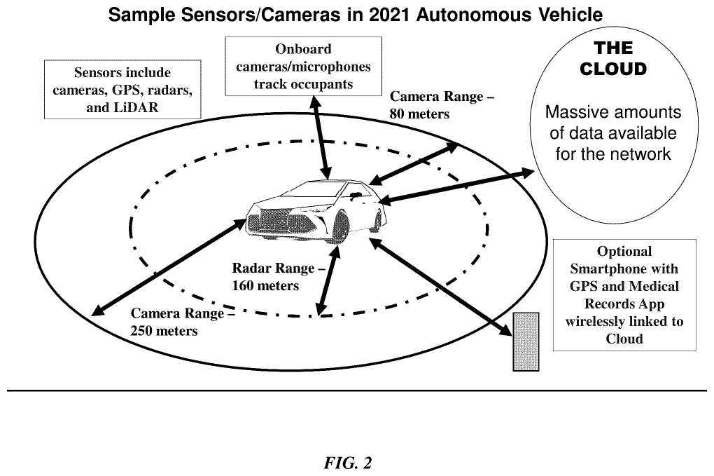

FIG. 2 shows that autonomous vehicles use a large number of cameras, radars, and LiDAR to map the world around the vehicle and thereby plan the optimal, safe drive. In 2021, the industry leader Tesla used 8 cameras with 360 degrees of view with a range of up to 250 meters in front and 50 meters behind, while ultrasonic sensors enhanced the mapping around the car and radar even permitted seeing ahead even during rain, or in the fog or with dust.

However, internal cameras used to monitor the driver's condition have created controversy. These internal cameras record and upload to the cloud the driver's actions prior to a crash. While the user can opt-out of this option, this is creating privacy concerns. To address these privacy concerns and due to the limitations of facial and voice recognition software, the passengers in a vehicle could voluntarily disclose medical and other information to the vehicle's computers manually or via an automatic phone app when they enter the vehicle.

Automated Emergency Calls are Mandated in the EU and should be Required in the U.S.

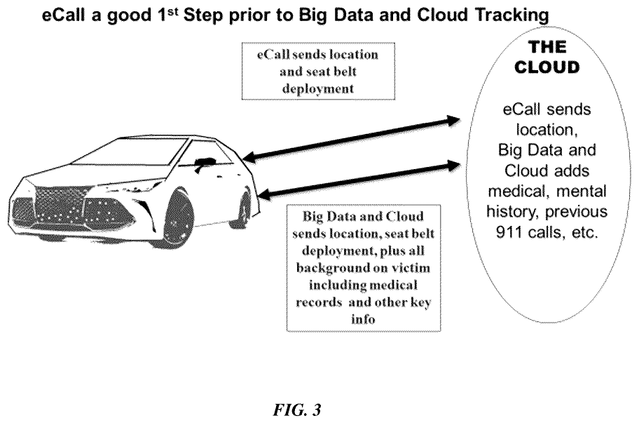

FIG. 3—Telematics is a tracking device in vehicles which sends key information such as location, speed and harsh braking or acceleration. Up to 10 percent of drivers in the US use this technology since insurance companies offer discounts for sharing this data. However, in 2021 the European Union was far ahead of the US since after April 2018 all European Car Makers have been required to include eCall, an automated emergency call technology. When a crash is occurring eCall automatically sends the location and also indicates if the airbags were deployed. However, using GPS data and other sensor information, Big Data with facial and voice recognition software identifies the driver's ID, medical history and all relevant information needed to treat the victim.

Track Me—Option in Sporadic Cell Phone Coverage Areas

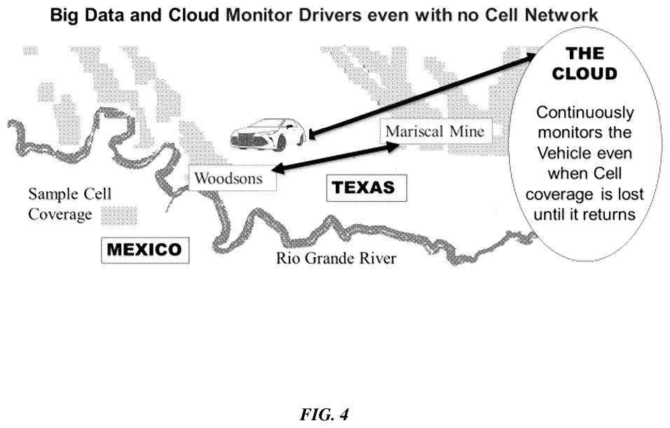

FIG. 4—One of the most remote areas in the lower 48 states is the Frank Church-River of No Return Wilderness in Idaho which has 2.37 million acres. It almost limited if any cell phone coverage. For example, Dixie Idaho (2019 population, 3,237) is near this area had limited cell coverage (2019). Large areas in Alaska have almost no coverage. The drive from Woodsons, Texas which is inside Big Bend National Park, to the Mariscal Mine, which is also in Big Bend, illustrates the problem and solution. This 11-mile drive takes roughly 55 minutes and has sporadic cell coverage. In Big Bend, wilderness areas and many National Parks, there should be the telematics option to turn on an optional Track Me where the system monitors the status of those vehicles which have lost cell coverage. After a pre-determined time, it would make an automatic phone call to confirm that the driver was OK. And then after a specified time even a 911 call could be made. This option could also be valuable when there was equipment failure on-board the vehicle or a breakdown of part of a cell network.

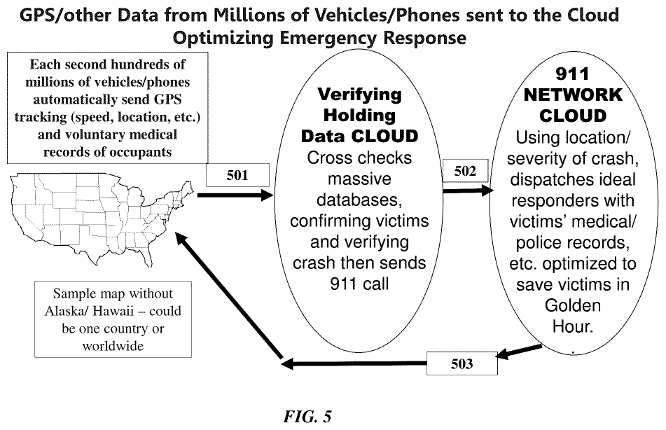

FIG. 5. 501—The system sends info on millions of vehicles to the cloud optimizing the emergency response. The vehicle and/or phone app automatically sends its GPS location, speed, acceleration, and photos/voice and/or IDs of occupants plus any voluntary medical ID provided by the occupant. A phone can provide redundant GPS data which confirms the crash to the cloud in order to minimize the number of incorrect 911 calls. The cloud cross checks massive databases, including crash data and vehicles' crash data histories identifying occupants and optimizing response to save victims in Golden Hour.

GPS data can be sent in small packets of only 80 characters per second using a smart phone and/or computers on a vehicle. While additional data complements the system, the GPS data is sufficient to confirm the location of the crash and its severity including the crash speed, etc. Since the medical records only change at most every 10 minutes, assuming people entering or exiting the vehicle, the average size of the key medical data is less than 40 characters per second. Therefore only 120 bytes of data per second is needed. However, the number of bytes needed and the time interval could be adjusted to expedite EMTs response times based on network storage and bandwidth issues. Over time, with the massive amounts of information, more data beyond GPS and medical information could be sent if this improved emergency response times.

In 2021, per the US Department of Transportation there were 276 million vehicles in the U.S. including 156 million trucks, 108 million cars, 8.5 million motorcycles, and 575 thousand buses. Statista data estimated that the USA has 280 million smartphone users in 2020. Redundancy in data sources reduces incorrect 911 calls. For example, if a person carried a cell phone on a motorcycle and dropped it this indicates a crash, however if there were an onboard tracking device on the motorcycle it would confirm the motorcycle didn't crash avoiding an unnecessary 911 call.

Sending 120 bytes of GPS and medical data per second is low bandwidth for any device on a network, such as a 5G network. With millions of vehicles and phones, even small data packets on the cloud create hundreds of Gigabytes of data per second or Terabytes each hour. For privacy concerns and to reduce storage requirements, this data could be constantly discarded since the only relevant data is actual crash and/or emergency data. FIG. 5 502 shows the data sent to a queue which uses AI (artificial intelligence and/or statistics), to verify that a crash has occurred. Finally, FIG. 5 503 the cloud reconfirms the GPS location and severity of crash, and dispatches ideal responders with victims' medical records, etc. optimized to save victims in Golden Hour.

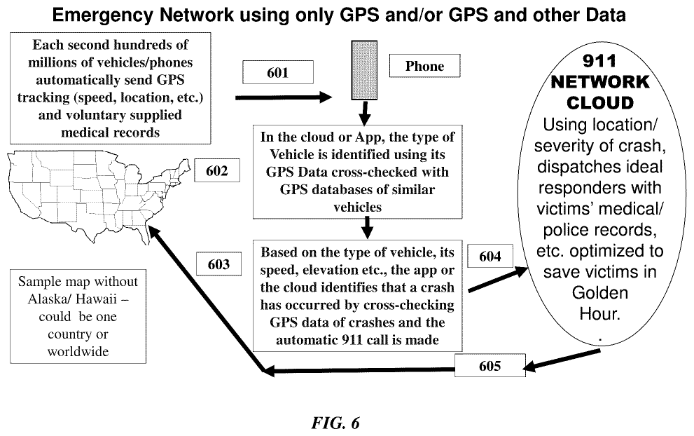

FIG. 6 shows the system works using only GPS data from a vehicle and/or cell phones FIG. 6 601. In 2022, most vehicles lacked the advanced electronics to automatically send GPS data. A person can make a manual 911 call however the best solution is this system where a cell phone app automatically calls 911 with the GPS location data comprising the speed and severity of the crash. An automatic 911 call would be made even if the occupant of the vehicle was unable to call. With only GPS data, the system cross-checks historical databases GPS profiles of similar vehicles to confirm the type of vehicle, i.e. a car, train, bus or plane since the vehicle GPS data and crash data of each vehicle is different FIG. 6 602 and then uses the speed and other parameters of that vehicle determines if a crash has occurred FIG. 6 603. The system uses AI and/or statistical data to minimize unnecessary 911 calls. The first priority would be to respond to calls which had redundancy with multiple sensors FIG. 6 604. The network could give a lower priority to 911 calls generated from only cell phone generated calls to avoid overloading the 911 system FIG. 6 605.

FIG. 6 comprises continuous coverage of GPS tracking, however due to privacy concerns, the users could opt in to tracking, such as automatically when the person enters a vehicle, or for children who leave their home. Ultimately, based on Big Data collected overtime, AI could determine to make a 911 call and the system could be adjusted to limit tracking to only vehicles if too many incorrect 911 calls were made. The longer Big Data collects and analyzes millions of smartphone and vehicle data for crashes it increasingly eliminates incorrect 911 calls.

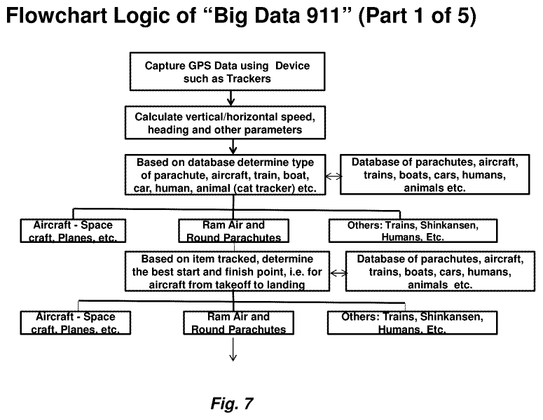

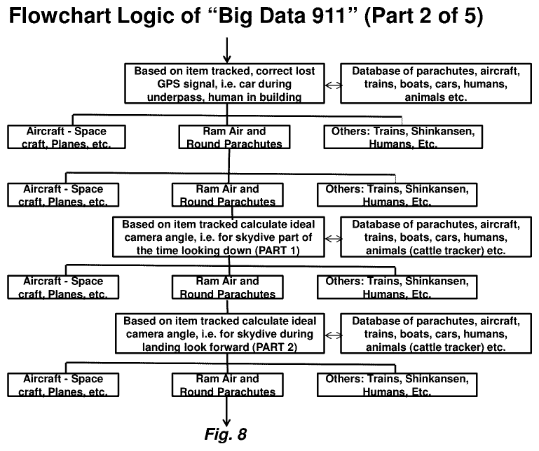

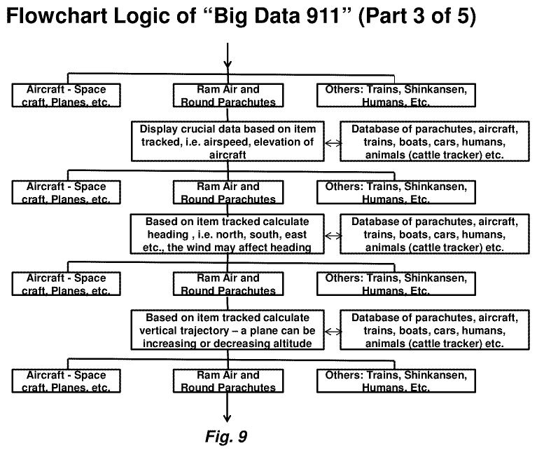

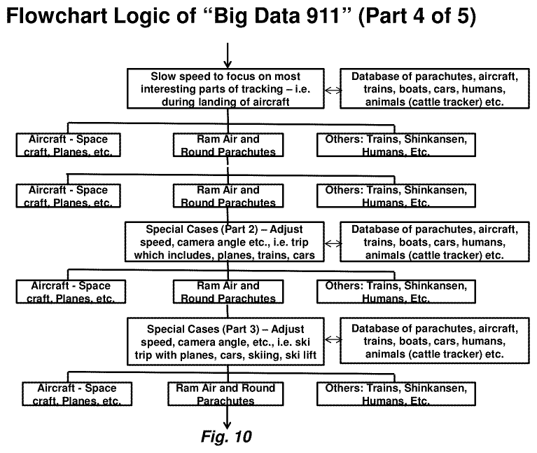

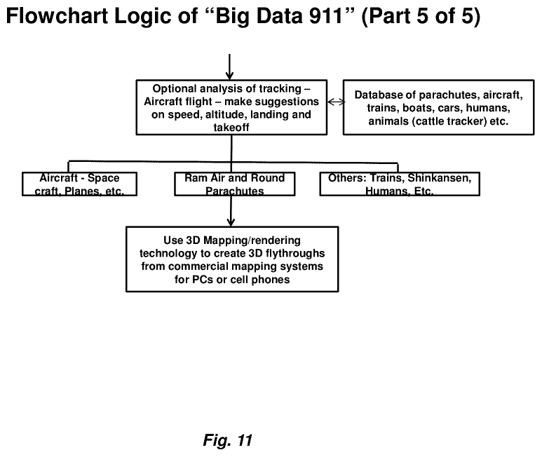

FIG. 7-11 show how the tracker data is used to identify the type of movement, i.e. aircraft, skydive etc., and correcting GPS data errors, such as lost data or corrupted data, and calculating the best angles and perspectives to view the plots in 3D interactive maps or videos which could be used for debriefings during accident investigations. FIG. 7 summarizes how the captured data is cross-checked with a database of many types of movements, from humans to trains, Round and Ram Parachutes, helicopters, Propeller Aircraft, Jet Aircraft and Spacecraft. Then the 3D interactive maps with the best angles for debriefings and accident investigations are automatically created.

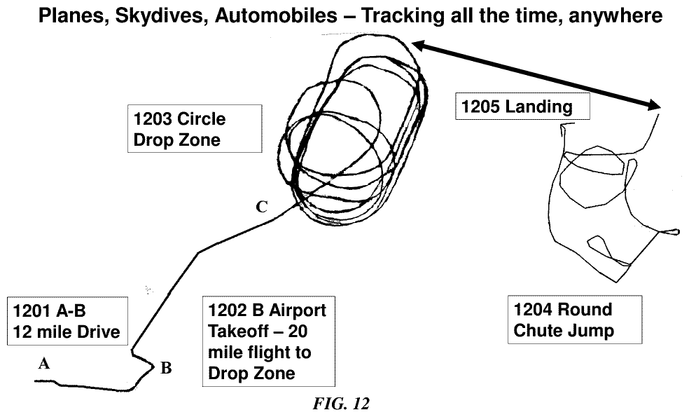

FIG. 12 summaries Big Data 911's amazing capabilities. It has tracked hundreds of skydives from a person's home until the skydive landing. It detected potential emergencies for a person who drove from their home (FIG. 12 1201) to an airport, boarded a plane (FIGS. 12 1202-1203), and skydived using a Round or RAM parachute. (FIGS. 12 1204-1205). The actual debriefing tracking is shown on a 3-D color map—however patent applications are limited to 2-D black and white. Using only error corrected GPS data which is cross-checked with historical data for the type of vehicle, it indicates if there is a crash or emergency in a car, plane or during skydives. It's ideal for those who fly small planes, drive older cars, skydive, and/or engage in extreme sports in remote locations, where Advanced Automatic Collision Notification (AACN), the industry standard for determining crashes, may not be available.

For tracking, Big Data 911 only requires a GPS enabled cell phone, or a similar device such as a satellite phone. Technologies such as Starlink satellites permit standard cell phones to communicate literally anywhere, extending tracking everyone all the time, anywhere. Big Data 911 can save hundreds of lives, roughly 1 life per million phones tracked as noted below, however to do this it must track all phones all the time. Also, GPS has major limitations such as lost signals in tunnels, canyons or in buildings where there isn't a line of site to the GPS satellites, therefore GPS must be error correct to reflect lost signal and other anomalies. Also, phones are turned off, fail and with more sensors they more quickly run out of power. If all of these anomalies aren't cross-checked with historical data in the cloud, there could be too many unnecessary 911 calls. However, attention should be considered for cell phones in remote areas, or late at night when no witness might report an accident. In those cases, more leeway should be given since a safety check could be worth the risk of an unnecessary 911 call.

Hundreds of Lives saved each year—AACN uses (1) sensors such as accelerometers, air bag deployment etc. to detect crashes and (2) it uses GPS for location only, not to determine where crashes occur. While AACN is powerful—it's more expensive requiring multiple sensors which drain power. Also, GPS without error-correcting doesn't provide accurate enough data. Big Data 911 is unique because it only needs error-corrected GPS's calculated acceleration/deceleration to determine a crash. The US DOT (AACN Research Report (No. DOT HS 812 729), 2019, May) estimated AACN saves 360 where AACN's benefits exceeded costs by $2.18 billion. Big Data 911 using only GPS data could save lives when there's no AACN on a vehicle, or in other situations such as skydives. Assuming that up to 33% of vehicles either lacked AACN or Big Data 911 provided redundancy predicting crashes, Big Data 911 could save 120 lives per year, a net benefit of $0.72 billion.

Other Issues: Worldwide, Big Data 911 could track billions of phones and vehicles. As noted, to make the system manageable, GPS data alone confirms the type of vehicle (plane, car, etc.), and if a crash occurred. Redundancy improves the system since data from a phone could complement/reconfirm vehicle data. In 2014, they spent hundreds of millions of dollars to find Malaysian Flight 370 which stopped communications and crashed. If a passenger's satellite cell phones were on, the location of the plane could be continuously tracked.

The system would be constantly discarded data at the end of a safe vehicle trip, such as when a plane landed. Nevertheless, the amount of data would be massive. Data centers in various parts of the world could work together to share GPS and other data. For example, the EU could handle European travel, the US would handle North America, etc. A final key point is privacy—the user would opt in to disclose their GPS data. Companies, such as Google, collect massive amounts of data for ads, and some have suggested TicTok is being used to collect data for the Chinese government. On the other hand, data collected for Big Data 911 would be used to save lives by identifying accidents. And auto insurers could give incentives for those without GPS vehicle trackers to simply keep their phone on while they drive, not to sell ads, but to protect their safety.

What is claimed is:

1. A method comprising: sending GPS and other data to the cloud from a vehicle's sensors and cameras and/or from a smartphone's sensors, and automatically detecting a vehicle crash and/or other emergency using GPS and other data, cross-checking with historical crash data and automatically calling 911 with all crash information, comprising GPS location and severity of the crash, and sending the victim's medical records and caller ID which are collected using onboard cameras and sensors to identify the victims and/or with medical information voluntarily linked to the cloud via a phone where this medical and GPS data ensures the optimum medical response during the crucial Golden Hour after the accident and that EMTs (Emergency Medical Technicians), police and other responders provide the optical medical treatment both en route to the hospital and once there where the system's Big Data AI or statistical information expedites the ideal medical solution for the victim and this method could be used nationally or internationally to safely track a person traveling worldwide.

2. The method of claim 1 further comprising: using the vehicle's video and/or audio, or voluntarily uploaded app data to the vehicle's computers, confirming the caller's ID to retrieve prior medical history, police reports including criminal records and mental history which indicates police may be needed, and comprising all relevant data in order to immediately begin the appropriate care and response associated with the incident-type profile for the call; and using Advanced Automatic Collision Notification (AACN) and the injury severity score (ISS) to improve post-crash medical care using Big Data in the cloud to monitor millions of vehicles and/or individuals in one country or worldwide as the person travels so that their medical history is immediately available to provide the optimal treatment after accidents.

3. A system comprising: a communication unit, a GPS unit, with an optional link to a vehicle's on-board sensors to continually monitor from the cloud and store the vehicle's GPS location, acceleration, deceleration, elevation, speed and the status of the vehicle's systems in the cloud and cross checking historical crash data in the cloud to determine if an emergency condition exists including a crash, fire or other emergency and to then sending a 911 distress call to request the dispatch of EMT and/or police to the incident with all related medical information and other records of the potentially injured party.

4. A system wherein a victim is identified using one or more of the following: a phone's caller ID, a vehicle's cameras and facial and/or voice recognition software and this info is cross-checked with the cloud and the option to voluntarily provide medical records linked via a phone app to the vehicle's computers to generate and update the accident victim's profiles based on one or more of data obtained in association with the call and the historical data, the historical data further comprising one or more of: previous call data; police records; jail data; social media data; medical records; security records; and customer records.

5. The method of claim 1, further comprising, tracking all vehicles which have lost vehicle and/or cell contact with the cloud and after a time period selected by the driver, automatically calling and/or texting the driver confirming their status, and if this safety call or text fails, calling 911 if the loss of cell coverage exceeds a preset time limit.

6. The method of claim 1, further comprising a smart phone or satellite phone app and/or software linked to the cloud which continuously sends the phone's GPS data to track the vehicle and/or person and confirm when crashes occur using GPS elevation, acceleration and deceleration data cross-checked with databases of prior crashes of similar vehicles thereby providing the cloud accurate crash and/or other emergency notification and avoiding incorrect 911 calls.

7. The method of claim 1, further comprising tracking via GPS data and consensually shared medical records from phone apps and/or vehicle sensors expediting medical help from responders after crashes or other emergencies where the redundancy of the tracking devices ensures that automatic calls to the 911 network occur in an emergency and not due to lost and/or corrupt GPS and other data and/or equipment malfunctions.

8. A system which works worldwide comprising: GPS data from a smartphone or satellite phone which tracks the vehicle and its occupants and corrects the GPS data for anomalies including lost or corrupted GPS signals, or loss of power, by cross-checking with prior GPS data creating clean GPS data which it shares with the cloud which it uses to calculate the related elevation, speed, acceleration/deceleration and other data confirming the type of vehicle tracked, including all types of vehicles such as planes, trains, motorcycles, cars, etc., and when the vehicle crashes and/or other emergency occurs and storing this data in the cloud to continuously improve the analysis of vehicle crash data to reduce the chances of incorrect 911 calls and when a crash is detected the system makes an automatic call and/or sends a distress text message to the 911 network requesting emergency responders and finally after a crash it plots the crash on 3D maps, such as Google Earth, for debriefing during accident investigations.
